(19) United States
(12) Patent Application Publication
(10) Pub. No.: US 2015/0240251 A1
(43) Pub. Date: Aug. 27, 2015

(54) COMPOSITION FOR REGULATING FLOWERING OF A PLANT CONTAINING THE GENE CODING FOR ABI3 PROTEIN

(71) Applicant: POSTECH ACADEMY-INDUSTRY FOUNDATION, Gyeongsangbuk-do (KR)

(72) Inventors: Il Doo Hwang, Pohang-si (KR); Ho Jin Ryu, Chungcheongbuk-do (KR); Won Sil Bae, Pohang-si (KR)

(21) Appl. No.: 14/427,940

(22) PCT Filed: May 10, 2013
(86) PCT No.: PCT/KR2013/004183

§ 371 Date: Mar. 12, 2015

(30) Foreign Application Priority Data
Sep. 13, 2012 (KR) 10-2012-0101641

(51) Int. Cl. C12N 15/82; G01N 33/566; C07K 14/415

(52) U.S. Cl. CPC C12N 15/827; C07K 14/415; C12N 15/8205; G01N 33/566; G01N 2333/415

(57) ABSTRACT

Provided are a composition containing ABI3 gene and a method of delaying flowering of plants using the same. The method has an excellent effect on delaying of flowering of plants. In the previously known method of delaying the flowering of plant, since flowering is delayed by silencing the expression of a gene for promoting flowering, it is difficult to regulate a degree of delaying of flowering. Also, since a gene silencing method such as RNAi is used, it is not easy to produce an actual transgenic plant and thus it is difficult to use for industry. However, ABI3 gene acts in a manner which delays flowering when expression increases, and thus it is easy to regulate the degree of delay in flowering and to produce a transgenic plant. Thus, it is expected that the present disclosure can be effectively applied to increase the productivity of crops by delaying flowering time or blocking flowering in plants of which vegetative tissue or leaves are used.

### TECHNICAL FIELD

The present application relates to a composition for regulating flowering of a plant, which includes a gene coding for ABI3 protein, and a method of delaying or repressing flowering of a plant using the same.

### BACKGROUND ART

Plant flowering is a changing point from vegetative growth to reproductive growth, and is very important in agricultural aspects. In the case of leaf vegetables in which leaves of the plant are used for food (e.g., vegetables such as sesame leaves, lettuce, etc.), when flowering occurs, vegetative growth no longer progresses and every energy is focused on fruiting. Therefore, farms have to input a lot of labor power to delay the flowering through artificially regulated day length and environmental change. In addition, in the case of crops using vegetative tissues accumulated in roots and stems (e.g., potato, garlic, onion, sweet potato, white radish, etc.), when flowering occurs, nutrients are no longer reserved in root tissues. Therefore, if the plant flowering is delayed or blocked, the labor power of the farms will be reduced, and crop yields will be increased according to an extended vegetative growth period of plants.

The plant flowering is a complex plant development program consisting of a variety of signal transduction. Such signals are recently known to be regulated by light, water, temperature and plant hormones. A flowering promoting effect of the plant hormone, gibberellin, is well known, and it is recently reported that brassinosteroid also has a flowering promoting effect (Domagalska et al., 2010, PLoS ONE, 5(11):e14012). However, it was reported that abscisic acid (ABA) serves as a complimentary role with the two hormones described above to delay flowering (Domagalska et al., 2010, PLoS ONE, 5(11):e14012). The ABA is known as a hormone mediating many reactions of a plant required to overcome stresses. Such a flowering delaying effect of the ABA well corresponds to the characteristic in which a plant is not flowered under a stress. However, there is almost no study for how to regulate flowering by signal transduction of these hormones on the molecular level.

Recently, according to the development of molecular genetics using Arabidopsis thaliana as a model plant, various genes involved in flowering are identified. However, most of them are positive regulatory factors for flowering which can delay the flowering when expression of genes is silenced. For example, in U.S. Pat. No. 6,225,530, a gene for regulating a flowering time of plants FT (FLOWERING LOCUS T), which is isolated from Arabidopsis thaliana, a polypeptide encoded by the FT and a method of regulating the flowering time of plants using the FT gene are disclosed. The FT is also known as florigen. The expression of the FT is regulated by various flowering regulatory factors through complex interactions. External signals such as light, temperature and photoperiod and internal signals such as nutrient condition and hormones are involved in flowering, and a flowering pathway is largely divided into a photoperiod pathway, a vernalization pathway, a gibberellin (GA) pathway, and an intrinsic pathway. When the expression of the FT is increased, flowering is promoted, and when the expression of FT is repressed, the flowering time is delayed. Accordingly, to delay the flowering time using the FT, gene silencing has to be performed using RNAi or microRNA. However, such techniques have serious disadvantages in which other genes having similar genetic information existing in a plant are also silenced, and thus are difficult to be applied in practice.

In the middle of a study on genes isolated from Arabidopsis thaliana, and involved in a flowering time regulating mechanism, the inventors found that, when ABA INSENSITIVE 3 (ABI3) gene is overexpressed in the plant of Arabidopsis thaliana, flowering is delayed. They also found that, in the overexpression of ABI3, a flowering promoting effect caused by reinforcement of brassinosteroid signal transduction also overcome.

It has been reported from a previous study that ABI3 gene positively regulates ABA signal transduction in which plant seeds repress germination (Giraudat et al., 1992, Plant Cell, 4:1251-1261). It is known that ABI3 directly induces transcription of a positive regulatory transcription factor ABI5 mediating an ABA signal in the germination of seeds (Lopez-Molina et al., 2002, Plant Journal, 32:317-328). However, the delay of flowering found in the present disclosure is a new effect of the ABI3 gene, which has not been found so far.

Thus, the inventors completed the present disclosure relating to a composition containing ABI3 gene and a method of delaying flowering using the same.

### DISCLOSURE

### Technical Problems

The present disclosure is contrived to solve conventional technical problems described above, and is directed to providing a composition for delaying or repressing flowering of a plant containing a base sequence coding for ABI3 protein and a method of delaying or repressing flowering of a plant by overexpressing ABI3 gene in the plant using the same. The present disclosure is also directed to providing a method of selecting a flowering time regulating gene or protein using a transgenic plant or a plant cell in which ABI3 gene is overexpressed or the expression of ABI3 is repressed.

However, the technical problems of the present disclosure are not limited to the above-described problems, and other problems not described will be clearly understood by those of ordinary skill in the art from the following description.

### Technical Solution

In one aspect, the present disclosure provides a composition for delaying or repressing flowering of a plant containing a base sequence coding for an amino acid sequence of SEQ. ID. NO: 3. Here, the amino acid sequence of SEQ. ID. NO: 3 constitutes ABI3 protein.

In one embodiment of the present disclosure, the base sequence is a base sequence of SEQ. ID. NO: 1 or 2. SEQ. ID. NO: 1 denotes a gDNA sequence of the ABI3 gene, and SEQ. ID. NO: 2 denotes a cDNA sequence of the ABI3 gene. Meanwhile, due to degeneracy of codons, mutation in the base sequence does not bring about the change in protein. Accordingly, it is apparent to those of ordinary skill in the art that the base sequence used in the present disclosure is not limited to the base sequences of SEQ. ID. NOs: 1 and 2 described in the accompanying sequence list.

Here, the term “delay of flowering” means when the flowering is belated compared to a flowering time of a wild-type plant when cultivation conditions such as a temperature and day and night lengths are equal.

In another aspect, the present disclosure provides a composition for delaying or repressing flowering of a plant containing a recombinant vector for plant expression into which a base sequence coding for ABI3 protein is inserted. A type of vector used in transformation of a plant, a part of a plant or a plant cell is not particularly limited, and a vector generally used in the transformation of a plant, specifically, a vector such as pCB302ES or pGA1611, may be used.

In one embodiment of the present disclosure, the recombinant vector has a base sequence coding for ABI3 protein operably linked to a potent promoter and/or an enhancer which can be operated in a plant. However, a type of sequence for promoting the expression of ABI3 gene is not limited thereto, and may include all of a leader sequence, a transcription initiating sequence, a transcription terminating sequence, a replication origin, and a ribosome-binding site, which can have an influence on the expression of other linked genes. The “operably inserted” means that one is inserted such that transcription and/or translation of a gene is influenced by. For example, if a promoter has an influence on the transcription of a gene inserted together, it is considered that the gene is operably inserted.

The promoter sequence can be used in all of an inducible promoter sequence and a constitutive promoter sequence. The constitutive promoter may be, for example, a CaMV promoter, a CsVMV promoter, or the nopaline synthase (NOS) promoter, and the inducible promoter (a promoter possible to actively express a gene linked to an inducing factor in the existence thereof) may be, for example, a yeast metallothionein promoter activated by copper ions (Mett et al., 1993, Proc. Natl. Acad. Sci. USA, 90:4567), In2-1 and In2-2 promoters activated by a substituted benzene sulfone amide (Hershey et al., 1991, Plant Mol. Biol., 17:679), a GRE regulating sequence regulated by glucocorticoid (Schena et al., 1991, Proc. Natl. Acad. Sci. USA, 88:10421), an ethanol regulatory promoter (Caddick et al., Nature Biotech., 16:177, 1998), a photoregulatory promoter derived from a small subunit of a ribulose bis-phosphate carboxylase (ssRuBisCO) (Coruzzi et al., 1984, EMBO J., 3:1671; Broglie et al., 1984, Science, 224:838), a mannopine synthase promoter (Velten et al., 1984, EMBO J., 3:2723), a nopaline synthase (NOS) and an octopine synthase (OCS) promoter, or a heat shock promoter (Gurley et al., 1986, Mol. Cell. Biol., 6:559; Severin et al., 1990, Plant Mol. Biol., 15:827). However, a type of promoter is not limited thereto. Meanwhile, the recombinant vector may additionally include a selection marker gene.

Here, the “marker gene” means a gene encoding a character for selecting a transformant containing such a marker gene. The marker gene may be an antibiotic-resistant gene or a herbicide-resistant gene. A suitable selection marker gene may be a gene for adenosine deaminase, a gene for dihydrofolate reductase, a gene for hygromycin-B-phosphotransferase, a gene for thymidine kinase, a gene for xanthine-guanine phosphoribosyltransferase, or a gene for phosphinothrisine acetyltransferase. However, the type of selection gene marker is not limited thereto.

In addition, the present disclosure provides a plant transformed by the composition. Specifically, the plant may be weeds in a farmland, food crops including rice, wheat, barley, corns, peas, potatoes, wheat, adzuki beans, oat, or sorghum, vegetables including Arabidopsis thaliana, Chinese cabbage, white radish, pepper, strawberry, tomato, watermelon, cucumber, cabbage, oriental melon, pumpkin, green onion, onion, or carrot, special crops including ginseng, tobacco, cotton, sesame, sugar cane, sugar beet, perilla, peanut, or canola, fruits including apple tree, pear tree, jujube tree, peach, kiwifruit, grape, tangerine, persimmon, plum, apricot, or banana, flowers including rose, gladiolus, gerbera, carnation, chrysanthemum, lily, or tulip, or forage crops including ryegrass, red clover, orchard grass, alfalfa, tall fescue, or Perennial ryegrass, but the type of plant is not limited thereto.

The composition of the present disclosure may be used to delay or regulate flowering of a plant of fruits and vegetables or flowers in accordance with a market situation and a weather situation. For example, grains are classified into three kinds including early variety, middle variety and late variety based on developing time to the flowering time after seeding. The early variety has small yields due to a short period of maturation of the plant, but is early harvested or shipped, and the late variety has the opposite advantages. Accordingly, it can be determined whether the early variety or the late variety is shipped using the composition of the present disclosure.

The most common plant to which the composition of the present disclosure is applied is sclerophyllous vegetables in which leaves and stems are aged rapidly and a market value highly decreases after the flowering, or bulb vegetables or root vegetables in which growth of a vegetative tissue such as a bulb or root is rapidly reduced when flowering occurs. The sclerophyllous vegetables may include sesame leaves, lettuce, Chinese cabbage, cabbage, bok Choy, spinach, crown daisy, leaf mustard, water parsley, chicory, young radish, green onion, bamboo shoots, and asparagus. The bulb vegetables may be garlic or onion. The root vegetables may include white radish, carrot, burdock, sweet potato, Japanese yam, and potato.

In still another aspect, the present disclosure provides a method of delaying or repressing flowering of a plant, which includes (A) transforming a plant by the composition; and (B) overexpressing ABI3 gene in the transformed plant.

In one embodiment of the present disclosure, the method may further include selecting a plant in which a flowering delay phenotype is induced.

In another embodiment of the present disclosure, step (A) includes manufacturing a recombinant expression vector by inserting a base sequence coding for ABI3 protein into an expression vector, transforming such a recombinant expression vector into Agrobacterium, and transforming a plant with the transformed Agrobacterium.

In yet another aspect, a method of selecting a flowering time regulating gene or protein using a transgenic plant or plant cell in which ABI3 gene is overexpressed or expression of ABI3 gene is repressed.

In one embodiment of the present disclosure, the method may include treating a candidate material to the transgenic plant or plant cell in which ABI3 gene is overexpressed or expression of ABI3 gene is repressed and measuring an influence on activity or expression of ABI3 gene.

### Advantageous Effects

A composition including ABI3 gene and a method of delaying plant flowering using the same have a strong effect on the delay of plant flowering. A conventionally known method of delaying flowering acts as a method of delaying flowering by silencing the expression of a gene promoting the flowering, and is difficult to adjust a degree of delaying the flowering. In addition, the conventionally known method uses gene silencing such as

RNAi, and is difficult to produce a real transgenic plant and also is difficult to be industrially used. However, when the expression of ABI3 gene of the present disclosure increases, the flowering is delayed, and therefore the degree of delaying flowering is easily regulated and the transgenic plant is easily produced.

### DESCRIPTION OF DRAWINGS

FIGS. 1A and 1B are images of representative one selected from wild-type Arabidopsis thaliana Columbia-0 (Col-0) and ABI3-overexpressed Arabidopsis thaliana at 32 days after seeding. FIG. 1C is an image of leaves obtained from the plants of FIGS. 1A and 1B until flowering.

FIG. 2 shows statistical results measured with 100 each of the wild-type Arabidopsis thaliana and ABI3-overexpressed Arabidopsis thaliana, and FIG. 2A shows a flowering time, and FIG. 2B shows the number of leaves required for flowering.

FIG. 3 shows that bes1-D mutant Arabidopsis thaliana is more early flowered and has a smaller number of leaves until flowering than the wild type En-2. However, in another brassinosteroid promoting mutant, bzr1-ID, flowering is delayed. En-2 and Col-0 are used as controls for bes1-D and bzr1-1D, respectively.

FIGS. 4A and 4B show expression levels of proteins when ABI3 and ABI5 genes are introduced to bes1-D mutant Arabidopsis thaliana, respectively, which are detected by western blotting. The expression level of ABI3 is high in four out of the five plants, and the expression level of ABI5 is high in two out of the five plants.

FIG. 5 is an image showing whether flowering occurs or does not occur at 25 days after germination from a bes1-D plant, a bes1-D ABI5 plant, and a bes1-D ABI3 plant. Lines identified in FIG. 4 that ABI5 is highly expressed were used.

FIG. 6 is a graph showing that the bes1-D ABI3 plant overcomes a phenomenon of advancing the flowering time due to the bes1-D mutation. Lines identified in FIG. 4 that ABI5 is highly expressed were used.

FIG. 7 shows that the bes1-D ABI3 plant overcomes a phenomenon of reducing the number of leaves until flowering due to the bes1-D mutation. Lines identified in FIG. 4 that ABI5 is highly expressed were used.

FIG. 8 shows whether aging is delayed or not in a 45-day-old ABI3-overexpressed plant.

### EMBODIMENT

Hereinafter, exemplary examples will be provided to help in understanding the present disclosure. However, the following examples are merely provided to easily understand the present disclosure, but the scope of the present disclosure is not limited to the following examples.

### EXAMPLE 1

### Experiment Materials and Methods

1-1. Plant Materials and Growth Conditions

All of wild-type Arabidopsis thaliana used herein are Columbia-0 variety (Col-0) and En-2 variety.

A plant, bes1-D (Yin et al., 2002, Cell, 109:181-191), in which BES1 gene used herein is mutated to promote brassinosteroid signal transduction, was generously provided from the lab of Professor Seunghwa Choi (Division of Life Science, Seoul National University). Since bes1-D variety is derived from En-2 variety, in Example using the bes1-D variety, the En-2 variety was used as a control.

Arabidopsis thaliana plants in which ABI3 and ABI5 genes are overexpressed were obtained by the method previously described by H. Sommer (Masiero et al., 2004, Development, 131:5981-5990). Simply explaining, the method of manufacturing a transgenic plant included injecting a transforming vector (pCB302ES) including ABI3 and ABI5 genes into Agrobacterium to raise until OD600 became 0.7 Abs, and inoculating the Agrobacterium to a flower of Arabidopsis thaliana (floral dip method).

The plant was grown in soil under a long day condition (16/8 hours (light/day)—light was provided at an intensity of 120 μmol m⁻¹s⁻¹) at 23° C.

1-2. Method of Measuring Flowering Time

A plant flowering time was represented as an average number of first leaves and days until the time for having flowers from at least 15 plants (time to bolting).

1-3. ABI3 DNA

While overexpressed lines were manufactured using cDNA (cABI3) of ABI3 gene in conventional experiments, in the present disclosure, overexpressed lines were manufactured using genomic DNA (gABI3) containing introns of ABI3 gene. That is, when an overexpressed transgenic plant was manufactured using gABI3 used herein, a phenotype of delaying flowering was shown.

A reason for using gDNA in the present disclosure is as follows: a gene of a multicellular eukaryotic organism consists of introns and exons actually coding for a protein, and it is known that, although genes are expressed in the same loci, various types of genes having differences in combination of amino acids of a protein are expressed, even in the same loci, due to alternative splicing in which a part of an intron is inserted into an exon according to various environments and changes during splicing. Therefore, the expression of all types of alternative genes specifically shown in development of a plant when gDNA is overexpressed can be expected, and thus it is anticipated that this will provide more accurate information to study characteristics of the genes.

### EXAMPLE 2

### Comparison of Flowering Times Between ABI3-Overexpressed Arabidopsis Thaliana and Wild-Type Arabidopsis Thaliana

2-1. Confirmation of Flowering at 32 Days After Growth and Number of Leaves Required for Flowering

One hundred each of ABI3-overexpressed Arabidopsis thaliana and wild-type Arabidopsis thaliana were grown, and checked after 32 days.

(1) Confirmation of Flowering

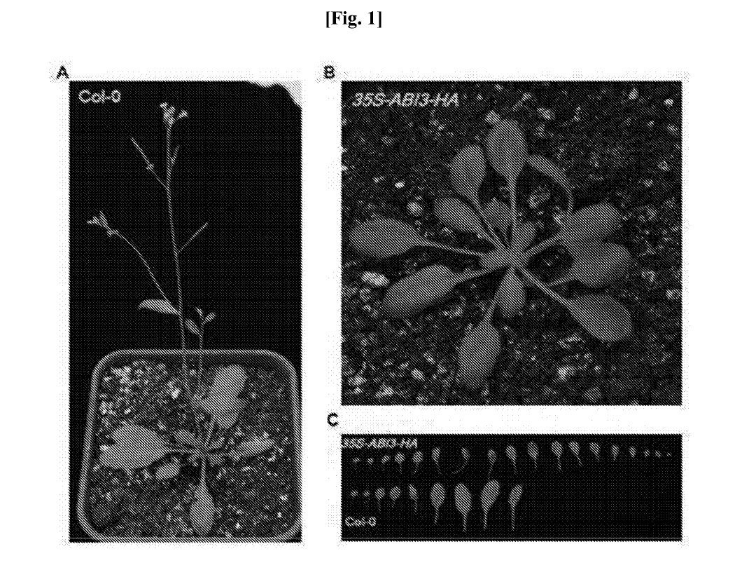

FIG. 1A is an image of representative one of wild-type Arabidopsis thaliana Columbia-0 (Col-0), which were grown for 32 days under the conditions described in Example 1-3, at flowering. FIG. 1B is an image of representative one of ABI3-overexpressed Arabidopsis thaliana (35S-ABI3-HA), which were grown for 32 days under the same conditions as described in FIG. 1A. As shown in FIG. 1B, flowering of the ABI3-overexpressed Arabidopsis thaliana was considerably delayed compared to that of the wild-type Arabidopsis thaliana, and transformants which were not flowered even at 32 days were 12% of the entire Arabidopsis thaliana (12 out of 100). These 12% Arabidopsis thaliana were not flowered after 60 days.

(2) Confirmation of the Number of Leaves

FIG. 1C is an image of leaves of the plant of FIG. 1A and the plant of FIG. 1B, which are arranged in an order of generation. As shown in FIG. 1C, from the wild-type Arabidopsis thaliana, 9 leaves were generated until flowering, and from the ABI3-overexpressed Arabidopsis thaliana, flowering was not induced even when 18 leaves (the number of leaves generated for 32 days after seeding) were generated.

2-2. Comparison of Flowering Time and the Number of Leaves Required for Flowering

(1) Confirmation of Flowering Time

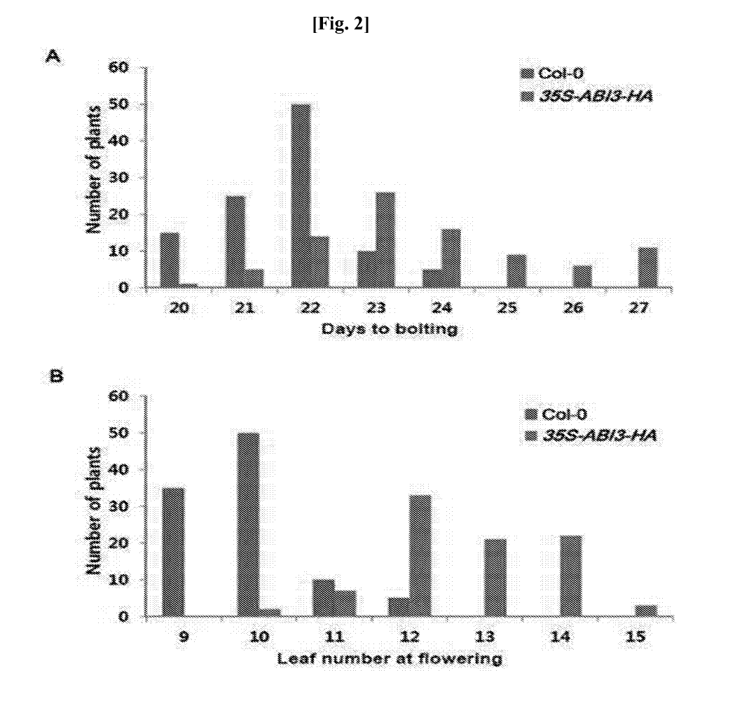

One hundred each of wild-type Arabidopsis thaliana and ABI3-overexpressed Arabidopsis thaliana were grown to estimate flowering time. The results are shown in FIG. 2A. As shown in FIG. 2A, flowering time of the wild-type Arabidopsis thaliana was 20 days for 14 plants, 21 days for 24 plants, 22 days for 52 plants, 23 days for 10 plants, and 24 days for 5 plants. Meanwhile, flowering time of ABI3-overexpressed Arabidopsis thaliana was 20 days for 1 plant, 21 days for 5 plants, 22 days for 14 plants, 23 days for 26 plants, 24 days for 16 plants, 25 days for 9 plants, 26 days for 6 plants, and 27 days for 11 plants, but in 12 ABI3-overexpressed Arabidopsis thaliana, vegetative growth for consistently forming leaves continued without flowering.

(2) Confirmation of the Number of Leaves

In addition, the number of leaves required for flowering was detected. The result is shown in FIG. 2B. As shown in FIG. 2B, in the case of the wild-type Arabidopsis thaliana, the number of leaves in flowering was 9 for 41 plants, 10 for 49 plants, 11 for 6 plants, and 12 for 4 plants. Meanwhile, in the case of ABI3-overexpressed Arabidopsis thaliana, the number of leaves was 10 for 2 plants, 11 for 7 plants, 12 for 33 plants, 13 for 21 plants, 14 for 22 plants, and 15 for 3 plants.

It can be seen from the results of Examples 2-1 and 2-2, in ABI3-overexpressed Arabidopsis thaliana, flowering time was delayed, and the number of leaves generated until flowering was considerably increased, compared to the wild-type.

### EXAMPLE 3

### Confirmation Whether ABI3 Overexpression was Offset Effect of Brassinosteroid Signal Transduction

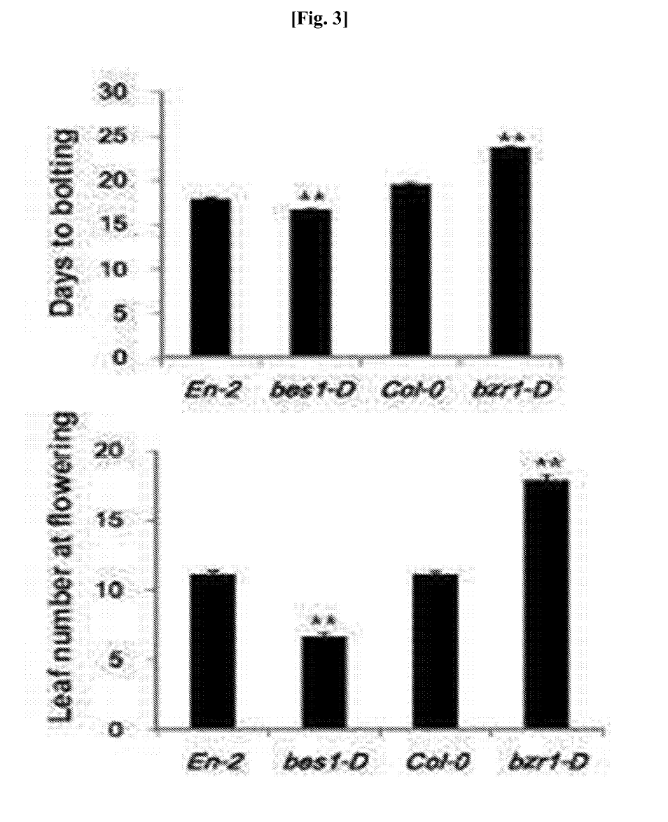

Flowering was promoted in a mutant, bes1-D, in which brassinosteroid signal transduction was promoted. FIG. 3 shows that, in bes1-D mutant Arabidopsis thaliana, flowering occurred earlier and the number of leaves until the flowering was smaller than those of the wild-type En-2. Meanwhile, bzr1-D is a mutant of BZR1 gene very similar to BES1 gene, and a plant in which brassinosteroid signal transduction is promoted like bes1-D. However, it can be confirmed from FIG. 3 that only the flowering of bes1-D was promoted, and the flowering of bzr1-D was delayed.

Accordingly, when the ABI3 gene was overexpressed in the bes1-D mutant Arabidopsis thaliana, to confirm whether a flowering promoting effect can be offset or not, the following experiments were performed.

3-1. Preparation of bes1-D Mutant Arabidopsis Thaliana in which ABI3 Gene and ABI5 Gene were Overexpressed

(1) Analysis of ABI3 Expression in Transgenic Arabidopsis Thaliana Plant

ABI5 is a gene directly induced to be expressed by ABI3 gene. Since a germination repressing effect of ABI3 is known as a phenomenon occurring by inducing the expression of ABI5, to check if the delay of flowering by ABI3 are caused by an increase in expression of ABI5, the case of overexpressing ABI5 was also checked.

To check the expression after the ABI3 gene and ABI5 gene were introduced to the bes1-D plant, the following experiment was performed.

A hemagglutinin (HA) epitope tag bound to a C-terminal end of ABI3 or ABI5 gene, and introduced to a bes1-D plant. An expression level of a protein in the plant was detected by western blotting using an anti-HA monoclonal antibody (Roche). For the experiment using a similar amount of a protein extract, an expression level of actin protein was used as a control. Here, the expression level of the actin protein used as a control was detected using an anti-actin monoclonal antibody (MP Biomedical).

In the drawing, the bes1-D plant to which ABI3-HA was introduced is represented as bes1-D 35S-ABI3-HA, and a plant to which ABI5-HA was introduced is represented as bes1-D 35S-ABI5-HA.

The “#” refers to a line. Here, each line is an independent transgenic plant. Even when the same transgenic vector was used, a gene to be overexpressed was randomly inserted into a genome of a plant. Accordingly, the independent transgenic plant has a different expression level of a gene according to a site of the genome of the plant into which the gene was inserted. Therefore, the function of the gene was identified by comparing at least two independent transgenic lines.

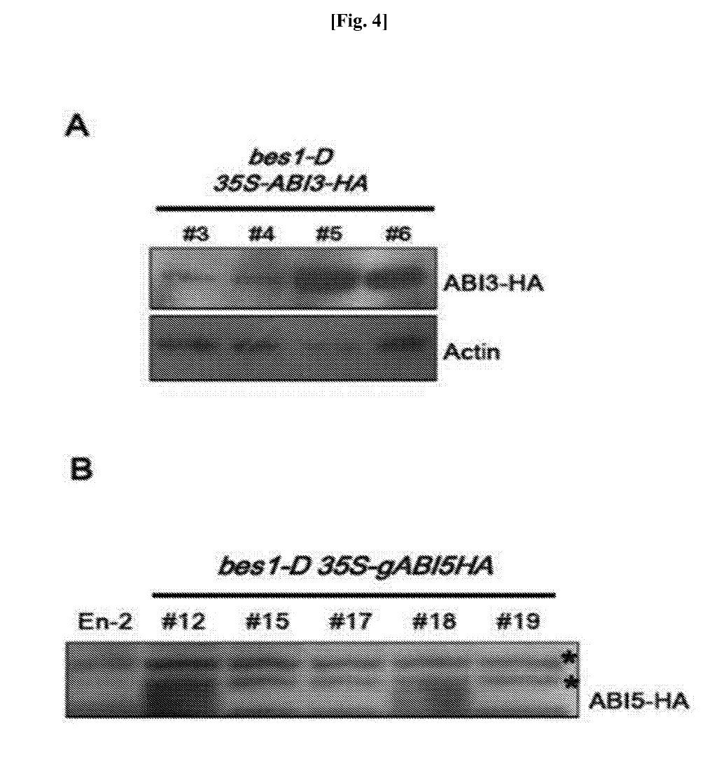

In FIG. 4A, four lines having high protein expression levels of ABI3 were represented. In FIG. 4B, lines having high expression levels of ABI5 protein were represented. It can be seen that, in #12 and #18 among the lines, the expression of the ABI5 protein was the highest.

3-2. Comparison of Flowering Between bes1-D Arabidopsis Thaliana, bes1-D ABI3 Arabidopsis Thaliana, bes1-D ABI5 Arabidopsis Thaliana

(1) Comparison of Flowering at 25 Days After Germination

The following experiment was performed using four lines (#3, #4, #5, #6) having the high expression levels of ABI3 protein and the #12 line having the highest expression level of ABI5 protein.

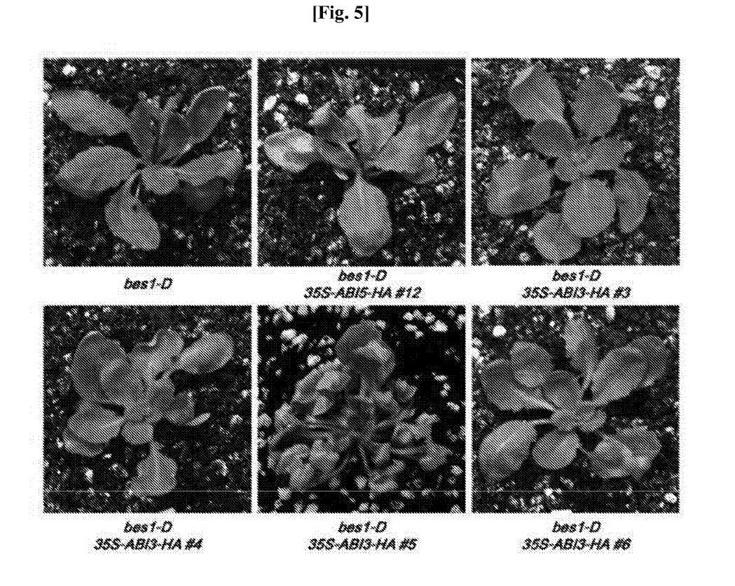

At 25 days after germination, bes1-D, bes1-D ABI5, and bes1-D ABI3 Arabidopsis thaliana were observed. As shown in FIG. 5, in the bes1-D and bes1-D ABI5 lines, flowering occurred, but in the bes1-D ABI3, flowering did not occur. Particularly, in the #5 line in which ABI3 was the most highly expressed, flowering did not occur, and vegetative growth continuously occurred (FIG. 5). Unlike when germination was repressed by ABI3, it can be seen that ABI5 was not involved in the delay of flowering.

(2) Comparison of Flowering Time

To obtain a statistical result for the result of FIG. 5, bes1-D mutant, and bes1-D ABI5 and bes1-D ABI3 transgenic Arabidopsis thaliana homozygote lines were planted in soil, and flowering time after germination was measured. Each independent transgenic plant selected in FIG. 4 (a transgenic plant having strong expression of a foreign protein) was used in an experiment (the number of independent transgenic plants used for statistical treatment was 14).

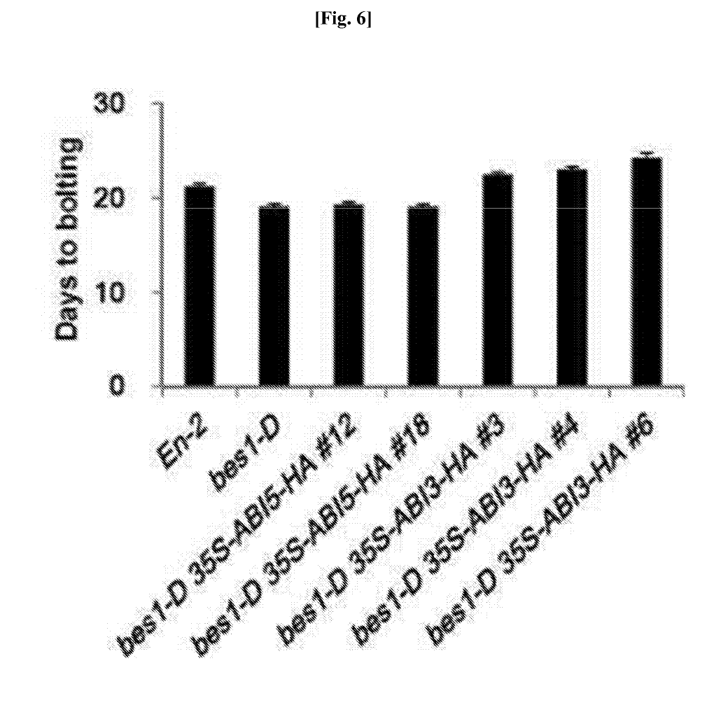

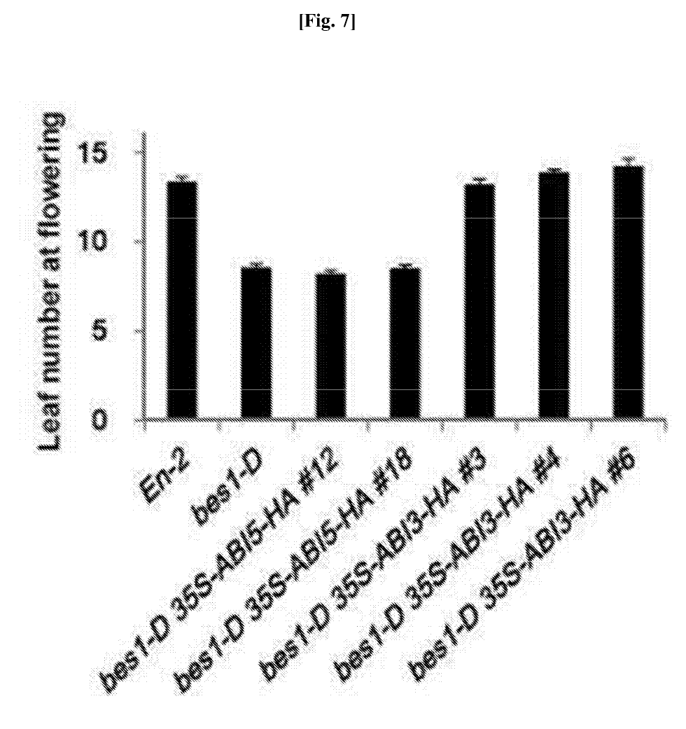

As shown in FIGS. 6 and 7, a flowering promoting effect caused by bes1-D mutation was offset only by ABI3 overexpression, but not by the overexpression of ABI5. Such results mean that only ABI3 has an effect of delaying the plant flowering.

(3) Comparison of the Number of Leaves Required for Flowering

Fourteen each of bes1-D, bes1-D ABI5 and bes1-D ABI3 Arabidopsis thaliana were seeded, and the numbers of leaves generated until flowering were observed. The results are represented as an average value of total 14 plants. As shown in FIG. 7, flowering occurred when a small number of leaves was observed due to bes1-D mutation, but the effect was offset by the overexpression of ABI3.

### EXAMPLE 4

### Confirmation of Delay of Aging of ABI3-Overexpressed Mutant

As the plant flowering occurred, a vegetative growth period is terminated, and due to a reproductive growth period, aging was performed. Accordingly, when the flowering was delayed by ABI3 overexpression, it was confirmed that the vegetative growth period is extended, and the aging of the plant was delayed.

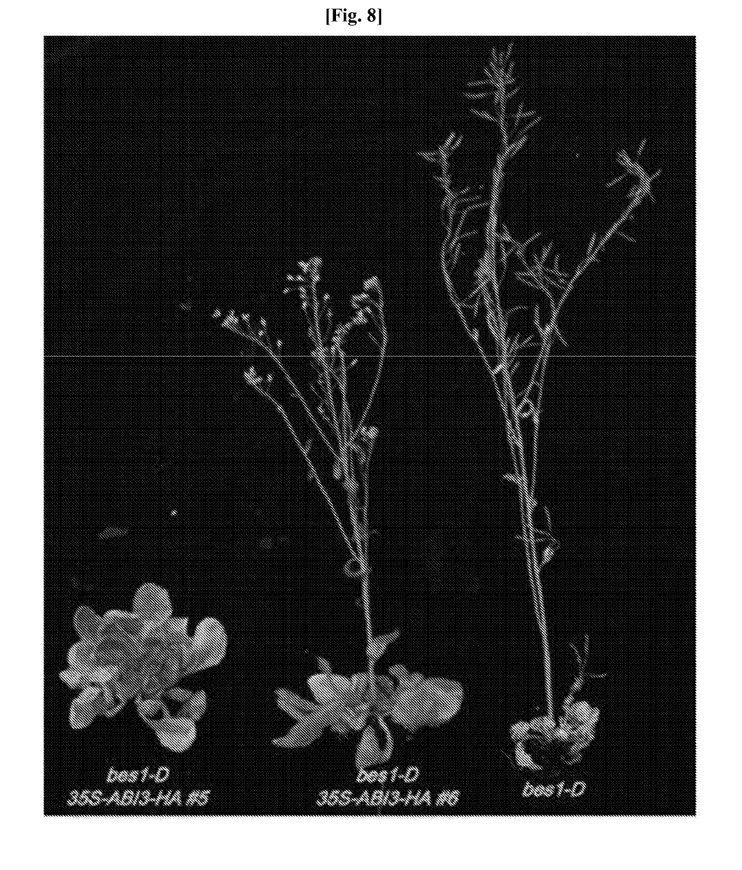

The ABI3-overexpressed lines were grown for 45 days, and the delay of aging was checked. In FIG. 8, it can be seen that the aging of the ABI3-overexpressed lines (#5 and #6 of the total four selected transgenic plants in which ABI3 gene was the most highly expressed) was delayed, compared to the bes1-D plant.

While the present disclosure has been shown and described with reference to certain embodiments thereof, it will be understood by those skilled in the art that various changes in form and details may be made therein without departing from the spirit and scope of the present disclosure as defined by the appended claims.

The present disclosure is expected to be critically applied to improve productivity of crops by delaying flowering time or blocking plant flowering using vegetative tissues or leaves.

| [Sequence List Text] | |
|---|---|
| <110> POSTECH ACADEMY-INDUSTRY FOUNDATION | |
| <120> Composition for controlling flowering time comprising gene | |
| encoding ABI3 protein | |
| <130> PCT01743 | |
| <150> KR 10-2012-0101641 | |
| <151> 2012-09-13 | |
| <160> 3 | |
| <170> KopatentIn 2.0 | |
| <210> 1 | |
| <211> 2870 | |
| <212> DNA | |
| <213> Artificial Sequence | |
| <220> | |
| <223> ABI3 gDNA | |
| <400> 1 | |
| atgaaaagct tgcatgtggc ggccaacgcc ggagatctgg ctgaggattg tggaatactc | 60 |
| ggtggagacg ctgatgatac tgttttgatg gatggaattg atgaagttgg tagagagatc | 120 |
| tggttagatg accatggagg agataataat catgttcatg gtcatcaaga tgatgatttg | 180 |
| attgttcatc atgacccttc aatcttctat ggagatctcc caacgcttcc tgatttccca | 240 |
| tgcatgtcgt cttcatcatc gtcttcaaca tctccagctc ctgtcaacgc aatcgtctcc | 300 |
| tcagcctctt cttcttcggc agcttcttcc tccacttcct cagctgcttc ttgggctata | 360 |
| ttgagatcag atggagaaga tccgactcca aaccaaaacc aatacgcatc aggaaactgt | 420 |
| gacgactctt ctggtgcatt gcaatccaca gcttccatgg agattccatt agacagcagt | 480 |
| caaggttttg gttgcggcga aggcggtggt gattgcattg atatgatgga gactttcggg | 540 |
| tacatggatc tacttgatag caacgagttc tttgacacct cagctatatt tagccaagac | 600 |
| gacgacacgc aaaaccctaa cttgatggac caaacccttg agagacaaga agaccaggtc | 660 |
| gttgttccga tgatggagaa taacagtggt ggagacatgc aaatgatgaa ttcttccttg | 720 |
| gaacaggacg atgatctcgc tgctgtgttt ttggagtggc taaagaacaa caaggagact | 780 |
| gtgtcggctg aggatttgag gaaagtaaag ataaagaaag ctacgattga atcagcggca | 840 |
| agaagactag gcggtggtaa agaagcgatg aagcagcttt taaagctgat tcttgaatgg | 900 |
| gtccaaacta atcacttaca aagaagacgc accaccacca ccaccaccaa cctctcttat | 960 |
| caacaatcat tccaacaaga tccatttcaa aaccctaacc ctaataacaa caacctaatc | 1020 |
| ccaccgtccg accaaacctg tttctcacct tcaacatggg ttcctccacc accacaacaa | 1080 |
| caagcttttg tctcggaccc gggttttgga tacatgcctg ctccaaacta tccgccacag | 1140 |
| ccagagttcc ttcctttact tgaatctcca ccgtcatggc caccaccacc acagtctggt | 1200 |
| cccatgccac atcaacaatt ccccatgccg ccaacctcgc agtataatca atttggagat | 1260 |
| ccaacaggtt tcaatggata caacatgaat ccgtaccaat atccttatgt tcctgcagga | 1320 |
| caaatgagag atcagagatt actccgtttg tgttcctcag caactaaaga ggcaagaaag | 1380 |
| aaacggatgg cgagacagag gaggttcttg tctcatcacc acagacataa caacaacaac | 1440 |
| aacaacaaca acaataatca gcagaaccaa acccaaatcg gagaaacctg tgccgcggtg | 1500 |
| gctccacaac ttaaccccgt ggccacaacc gccacgggag ggacctggat gtattggcct | 1560 |
| aatgtcccgg cagtgccgcc tcaattaccg ccagtgatgg agactcagtt acctaccatg | 1620 |
| gaccgagctg gctcagcttc tgctatgcca cgtcagcagg tggtaccaga tcgccggcag | 1680 |
| gtagacatgc acaatcatct ttctcattgt ttattcttta atttagcaat actcgaatag | 1740 |
| tttgtgtaat tgcaccactc gtttggtttg atgaagaagc taaagatttt acatgtgttt | 1800 |
| tttttgataa aatgtgaatc ggaagattat acatttgcaa tggaaatgaa atgattttga | 1860 |
| tctttttggt ttgattttag ggatggaaac cagaaaagaa tttgcggttt ctcttgcaga | 1920 |
| aagtcttgaa gcaaagcgac gtgggtaacc tcggaaggat cgttttgcca aaagtaattt | 1980 |
| ttcttctaat ttcttgtagc ctttgctttc cattttctaa aaaaggttca atgtttgtgt | 2040 |
| aaaaatattg tcaagttttt attttatttt tactcttatt ggttaagtta tattttactg | 2100 |
| aatttttatt ttttttagaa agaagctgag acacacttgc cggagctaga ggcaagagac | 2160 |
| ggcatctctc tggccatgga agacatcgga acctctcgtg tttggaacat gcgctacagg | 2220 |
| taactgatta tgatgctaac atgttaacat tgattctttt tataaaaaca attcgtgtat | 2280 |
| tttgtcaaaa attggaactc gaccaaaatg ttttttcggt tatttaattg tcttcttaaa | 2340 |
| ttggttttgc aggttttggc ctaacaacaa aagcaggatg tatctcctcg agaacaccgg | 2400 |
| tacgtttttg aaaatgtacc cgttaataat tttcctttct tttggtttgt ttgttctctt | 2460 |
| gtaattattg ttgtggacgc atacatatct aattttcctt gaaattacgt ttacaggcga | 2520 |
| ttttgtgaaa accaatgggc tccaagaagg tgatttcata gtcatatact ccgacgtcaa | 2580 |
| atgtggcaaa tatgtaagag aagcatcaca atatttttct atacttttca ttagtattta | 2640 |
| actctcatca ttacttttgt tggtatttat cttgtcataa ttaattgaga ataatattat | 2700 |
| gacagttgat acgaggggtt aaagtaagac aaccgagcgg acaaaagccg gaggccccac | 2760 |
| cgtcgtcagc agctacgaag agacaaaaca agtcgcaaag gaacataaac aataactctc | 2820 |
| cgtcggcgaa tgtggtggtc gcttcaccaa cttctcaaac tgttaaatga | 2870 |
| <210> 2 | |
| <211> 2163 | |
| <212> DNA | |
| <213> Artificial Sequence | |
| <220> | |
| <223> ABI3 cDNA | |
| <400> 2 | |
| atgaaaagct tgcatgtggc ggccaacgcc ggagatctgg ctgaggattg tggaatactc | 60 |
| ggtggagacg ctgatgatac tgttttgatg gatggaattg atgaagttgg tagagagatc | 120 |
| tggttagatg accatggagg agataataat catgttcatg gtcatcaaga tgatgatttg | 180 |
| attgttcatc atgacccttc aatcttctat ggagatctcc caacgcttcc tgatttccca | 240 |
| tgcatgtcgt cttcatcatc gtcttcaaca tctccagctc ctgtcaacgc aatcgtctcc | 300 |
| tcagcctctt cttcttcggc agcttcttcc tccacttcct cagctgcttc ttgggctata | 360 |
| ttgagatcag atggagaaga tccgactcca aaccaaaacc aatacgcatc aggaaactgt | 420 |
| gacgactctt ctggtgcatt gcaatccaca gcttccatgg agattccatt agacagcagt | 480 |
| caaggttttg gttgcggcga aggcggtggt gattgcattg atatgatgga gactttcggg | 540 |
| tacatggatc tacttgatag caacgagttc tttgacacct cagctatatt tagccaagac | 600 |
| gacgacacgc aaaaccctaa cttgatggac caaacccttg agagacaaga agaccaggtc | 660 |
| gttgttccga tgatggagaa taacagtggt ggagacatgc aaatgatgaa ttcttccttg | 720 |
| gaacaggacg atgatctcgc tgctgtgttt ttggagtggc taaagaacaa caaggagact | 780 |
| gtgtcggctg aggatttgag gaaagtaaag ataaagaaag ctacgattga atcagcggca | 840 |
| agaagactag gcggtggtaa agaagcgatg aagcagcttt taaagctgat tcttgaatgg | 900 |
| gtccaaacta atcacttaca aagaagacgc accaccacca ccaccaccaa cctctcttat | 960 |
| caacaatcat tccaacaaga tccatttcaa aaccctaacc ctaataacaa caacctaatc | 1020 |
| ccaccgtccg accaaacctg tttctcacct tcaacatggg ttcctccacc accacaacaa | 1080 |
| caagcttttg tctcggaccc gggttttgga tacatgcctg ctccaaacta tccgccacag | 1140 |
| ccagagttcc ttcctttact tgaatctcca ccgtcatggc caccaccacc acagtctggt | 1200 |
| cccatgccac atcaacaatt ccccatgccg ccaacctcgc agtataatca atttggagat | 1260 |
| ccaacaggtt tcaatggata caacatgaat ccgtaccaat atccttatgt tcctgcagga | 1320 |
| caaatgagag atcagagatt actccgtttg tgttcctcag caactaaaga ggcaagaaag | 1380 |
| aaacggatgg cgagacagag gaggttcttg tctcatcacc acagacataa caacaacaac | 1440 |
| aacaacaaca acaataatca gcagaaccaa acccaaatcg gagaaacctg tgccgcggtg | 1500 |
| gctccacaac ttaaccccgt ggccacaacc gccacgggag ggacctggat gtattggcct | 1560 |
| aatgtcccgg cagtgccgcc tcaattaccg ccagtgatgg agactcagtt acctaccatg | 1620 |
| gaccgagctg gctcagcttc tgctatgcca cgtcagcagg tggtaccaga tcgccggcag | 1680 |
| ggatggaaac cagaaaagaa tttgcggttt ctcttgcaga aagtcttgaa gcaaagcgac | 1740 |
| gtgggtaacc tcggaaggat cgttttgcca aaaaaagaag ctgagacaca cttgccggag | 1800 |
| ctagaggcaa gagacggcat ctctctggcc atggaagaca tcggaacctc tcgtgtttgg | 1860 |
| aacatgcgct acaggttttg gcctaacaac aaaagcagga tgtatctcct cgagaacacc | 1920 |
| ggcgattttg tgaaaaccaa tgggctccaa gaaggtgatt tcatagtcat atactccgac | 1980 |
| gtcaaatgtg gcaaatattt gatacgaggg gttaaagtaa gacaaccgag cggacaaaag | 2040 |
| ccggaggccc caccgtcgtc agcagctacg aagagacaaa acaagtcgca aaggaacata | 2100 |
| aacaataact ctccgtcggc gaatgtggtg gtcgcttcac caacttctca aactgttaaa | 2160 |
| tga | 2163 |

<210> 3
<211> 720
<212> PRT
<213> Artificial Sequence
<220>
<223> ABI3 protein
<400> 3
Met Lys Ser Leu His Val Ala Ala Asn Ala Gly Asp Leu Ala Glu Asp
1 5 10 15
Cys Gly Ile Leu Gly Gly Asp Ala Asp Asp Thr Val Leu Met Asp Gly
20 25 30
Ile Asp Glu Val Gly Arg Glu Ile Trp Leu Asp Asp His Gly Gly Asp
35 40 45
Asn Asn His Val His Gly His Gln Asp Asp Asp Leu Ile Val His His
50 55 60
Asp Pro Ser Ile Phe Tyr Gly Asp Leu Pro Thr Leu Pro Asp Phe Pro
65 70 75 80
Cys Met Ser Ser Ser Ser Ser Ser Ser Thr Ser Pro Ala Pro Val Asn
85 90 95
Ala Ile Val Ser Ser Ala Ser Ser Ser Ser Ala Ala Ser Ser Ser Thr
100 105 110
Ser Ser Ala Ala Ser Trp Ala Ile Leu Arg Ser Asp Gly Glu Asp Pro
115 120 125
Thr Pro Asn Gln Asn Gln Tyr Ala Ser Gly Asn Cys Asp Asp Ser Ser
130 135 140
Gly Ala Leu Gln Ser Thr Ala Ser Met Glu Ile Pro Leu Asp Ser Ser
145 150 155 160
Gln Gly Phe Gly Cys Gly Glu Gly Gly Gly Asp Cys Ile Asp Met Met
165 170 175
Glu Thr Phe Gly Tyr Met Asp Leu Leu Asp Ser Asn Glu Phe Phe Asp
180 185 190
Thr Ser Ala Ile Phe Ser Gln Asp Asp Asp Thr Gln Asn Pro Asn Leu
195 200 205
Met Asp Gln Thr Leu Glu Arg Gln Glu Asp Gln Val Val Val Pro Met
210 215 220
Met Glu Asn Asn Ser Gly Gly Asp Met Gln Met Met Asn Ser Ser Leu
225 230 235 240
Glu Gln Asp Asp Asp Leu Ala Ala Val Phe Leu Glu Trp Leu Lys Asn
245 250 255
Asn Lys Glu Thr Val Ser Ala Glu Asp Leu Arg Lys Val Lys Ile Lys
260 265 270
Lys Ala Thr Ile Glu Ser Ala Ala Arg Arg Leu Gly Gly Gly Lys Glu
275 280 285
Ala Met Lys Gln Leu Leu Lys Leu Ile Leu Glu Trp Val Gln Thr Asn
290 295 300
His Leu Gln Arg Arg Arg Thr Thr Thr Thr Thr Thr Asn Leu Ser Tyr
305 310 315 320
Gln Gln Ser Phe Gln Gln Asp Pro Phe Gln Asn Pro Asn Pro Asn Asn
325 330 335
Asn Asn Leu Ile Pro Pro Ser Asp Gln Thr Cys Phe Ser Pro Ser Thr
340 345 350
Trp Val Pro Pro Pro Pro Gln Gln Gln Ala Phe Val Ser Asp Pro Gly
355 360 365
Phe Gly Tyr Met Pro Ala Pro Asn Tyr Pro Pro Gln Pro Glu Phe Leu
370 375 380
Pro Leu Leu Glu Ser Pro Pro Ser Trp Pro Pro Pro Pro Gln Ser Gly
385 390 395 400
Pro Met Pro His Gln Gln Phe Pro Met Pro Pro Thr Ser Gln Tyr Asn
405 410 415
Gln Phe Gly Asp Pro Thr Gly Phe Asn Gly Tyr Asn Met Asn Pro Tyr
420 425 430
Gln Tyr Pro Tyr Val Pro Ala Gly Gln Met Arg Asp Gln Arg Leu Leu
435 440 445
Arg Leu Cys Ser Ser Ala Thr Lys Glu Ala Arg Lys Lys Arg Met Ala
450 455 460
Arg Gln Arg Arg Phe Leu Ser His His His Arg His Asn Asn Asn Asn
465 470 475 480
Asn Asn Asn Asn Asn Asn Gln Gln Asn Gln Thr Gln Ile Gly Glu Thr
485 490 495
Cys Ala Ala Val Ala Pro Gln Leu Asn Pro Val Ala Thr Thr Ala Thr
500 505 510
Gly Gly Thr Trp Met Tyr Trp Pro Asn Val Pro Ala Val Pro Pro Gln
515 520 525
Leu Pro Pro Val Met Glu Thr Gln Leu Pro Thr Met Asp Arg Ala Gly
530 535 540
Ser Ala Ser Ala Met Pro Arg Gln Gln Val Val Pro Asp Arg Arg Gln
545 550 555 560
Gly Trp Lys Pro Glu Lys Asn Leu Arg Phe Leu Leu Gln Lys Val Leu
565 570 575
Lys Gln Ser Asp Val Gly Asn Leu Gly Arg Ile Val Leu Pro Lys Lys
580 585 590
Glu Ala Glu Thr His Leu Pro Glu Leu Glu Ala Arg Asp Gly Ile Ser
595 600 605
Leu Ala Met Glu Asp Ile Gly Thr Ser Arg Val Trp Asn Met Arg Tyr
610 615 620
Arg Phe Trp Pro Asn Asn Lys Ser Arg Met Tyr Leu Leu Glu Asn Thr
625 630 635 640
Gly Asp Phe Val Lys Thr Asn Gly Leu Gln Glu Gly Asp Phe Ile Val
645 650 655
Ile Tyr Ser Asp Val Lys Cys Gly Lys Tyr Leu Ile Arg Gly Val Lys
660 665 670
Val Arg Gln Pro Ser Gly Gln Lys Pro Glu Ala Pro Pro Ser Ser Ala
675 680 685
Ala Thr Lys Arg Gln Asn Lys Ser Gln Arg Asn Ile Asn Asn Asn Ser
690 695 700
Pro Ser Ala Asn Val Val Val Ala Ser Pro Thr Ser Gln Thr Val Lys
705 710 715 720

1. A composition for delaying or repressing flowering of a plant, comprising: a base sequence coding for an amino acid sequence of SEQ. ID. NO: 3.

2. A composition for delaying or repressing flowering of a plant, comprising: a recombinant vector for plant expression into which a base sequence coding for an amino acid sequence of SEQ. ID. NO: 3 is inserted.

3. The composition according to claim 2, wherein, in the recombinant vector, the base sequence coding for an amino acid sequence of SEQ. ID. NO: 3 is operably linked to a strong promoter and/or enhancer which enables to be operated in plants.

4. The composition according to claim 2, wherein the recombinant vector further includes a selection marker gene which enables to check whether a plant is transformed or not.

5. The composition according to claim 1, wherein the base sequence is a base sequence of SEQ. ID. NO: 1 or 2.

6. A plant transformed using the composition according to claim 1.

7. The plant according to claim 6, wherein the plant is sclerophyllous vegetable, bulb vegetable or root vegetable.

8. The plant according to claim 6, wherein the sclerophyllous vegetable is selected from the group consisting of sesame leaves, lettuce, Chinese cabbage, cabbage, bok Choy, spinach, crown daisy, leaf mustard, water parsley, chicory, young radish, green onion, bamboo shoots, and asparagus.

9. The plant according to claim 6, wherein the bulb vegetable is selected from the group consisting of garlic and onion.

10. The plant according to claim 6, wherein the root vegetable is selected from the group consisting of white radish, carrot, burdock, sweet potato, Japanese yam, and potato.

11. A method of delaying or repressing flowering of a plant, comprising:
(A) transforming a plant by the composition according to claim 1; and
(B) overexpressing ABI3 gene by the transgenic plant.

12. The method according to claim 11, further comprising: selecting a plant from which a flowering delaying phenotype is induced.

13. The method according to claim 11, wherein, operation (A) includes manufacturing a recombinant expression vector by inserting a base sequence coding for an amino acid sequence of SEQ. ID. NO: 3 into an expression vector, transforming the recombinant expression vector in Agrobacterium; and transforming a plant using the transgenic Agrobacterium.

14. A method of selecting a flowering time regulating gene or protein using a transgenic plant or plant cell in which ABI3 gene is overexpressed or the expression of ABI3 gene is repressed.

15. The method according to claim 14, wherein the method includes treating a candidate material to the transgenic plant or plant cell in which ABI3 gene is overexpressed or the expression of ABI3 gene is repressed, and detecting an effect on the activity or expression of the ABI3 gene.


SEQUENCE LISTING (3 sequences)

SEQ ID NO: 1 - DNA, 2870 bp, Arabidopsis thaliana
  genomic DNA encoding ABI3 protein
atgaaaagcttgcatgtggcggccaacgccggagatctggctgaggattgtggaatactcggtggagacgctgatgatactgttttgatggatggaattgatgaagttggtagagagatctggttagatgaccatggaggagataataatcatgttcatggtcatcaagatgatgatttgattgttcatcatgacccttcaatcttctatggagatctcccaacgcttcctgatttcccatgcatgtcgtcttcatcatcgtcttcaacatctccagctcctgtcaacgcaatcgtctcctcagcctcttcttcttcggcagcttcttcctccacttcctcagctgcttcttgggctatattgagatcagatggagaagatccgactccaaaccaaaaccaatacgcatcaggaaactgtgacgactcttctggtgcattgcaatccacagcttccatggagattccattagacagcagtcaaggttttggttgcggcgaaggcggtggtgattgcattgatatgatggagactttcgggtacatggatctacttgatagcaacgagttctttgacacctcagctatatttagccaagacgacgacacgcaaaaccctaacttgatggaccaaacccttgagagacaagaagaccaggtcgttgttccgatgatggagaataacagtggtggagacatgcaaatgatgaattcttccttggaacaggacgatgatctcgctgctgtgtttttggagtggctaaagaacaacaaggagactgtgtcggctgaggatttgaggaaagtaaagataaagaaagctacgattgaatcagcggcaagaagactaggcggtggtaaagaagcgatgaagcagcttttaaagctgattcttgaatgggtccaaactaatcacttacaaagaagacgcaccaccaccaccaccaccaacctctcttatcaacaatcattccaacaagatccatttcaaaaccctaaccctaataacaacaacctaatcccaccgtccgaccaaacctgtttctcaccttcaacatgggttcctccaccaccacaacaacaagcttttgtctcggacccgggttttggatacatgcctgctccaaactatccgccacagccagagttccttcctttacttgaatctccaccgtcatggccaccaccaccacagtctggtcccatgccacatcaacaattccccatgccgccaacctcgcagtataatcaatttggagatccaacaggtttcaatggatacaacatgaatccgtaccaatatccttatgttcctgcaggacaaatgagagatcagagattactccgtttgtgttcctcagcaactaaagaggcaagaaagaaacggatggcgagacagaggaggttcttgtctcatcaccacagacataacaacaacaacaacaacaacaacaataatcagcagaaccaaacccaaatcggagaaacctgtgccgcggtggctccacaacttaaccccgtggccacaaccgccacgggagggacctggatgtattggcctaatgtcccggcagtgccgcctcaattaccgccagtgatggagactcagttacctaccatggaccgagctggctcagcttctgctatgccacgtcagcaggtggtaccagatcgccggcaggtagacatgcacaatcatctttctcattgtttattctttaatttagcaatactcgaatagtttgtgtaattgcaccactcgtttggtttgatgaagaagctaaagattttacatgtgttttttttgataaaatgtgaatcggaagattatacatttgcaatggaaatgaaatgattttgatctttttggtttgattttagggatggaaaccagaaaagaatttgcggtttctcttgcagaaagtcttgaagcaaagcgacgtgggtaacctcggaaggatcgttttgccaaaagtaatttttcttctaatttcttgtagcctttgctttccattttctaaaaaaggttcaatgtttgtgtaaaaatattgtcaagtttttattttatttttactcttattggttaagttatattttactgaatttttattttttttagaaagaagctgagacacacttgccggagctagaggcaagagacggcatctctctggccatggaagacatcggaacctctcgtgtttggaacatgcgctacaggtaactgattatgatgctaacatgttaacattgattctttttataaaaacaattcgtgtattttgtcaaaaattggaactcgaccaaaatgttttttcggttatttaattgtcttcttaaattggttttgcaggttttggcctaacaacaaaagcaggatgtatctcctcgagaacaccggtacgtttttgaaaatgtacccgttaataattttcctttcttttggtttgtttgttctcttgtaattattgttgtggacgcatacatatctaattttccttgaaattacgtttacaggcgattttgtgaaaaccaatgggctccaagaaggtgatttcatagtcatatactccgacgtcaaatgtggcaaatatgtaagagaagcatcacaatatttttctatacttttcattagtatttaactctcatcattacttttgttggtatttatcttgtcataattaattgagaataatattatgacagttgatacgaggggttaaagtaagacaaccgagcggacaaaagccggaggccccaccgtcgtcagcagctacgaagagacaaaacaagtcgcaaaggaacataaacaataactctccgtcggcgaatgtggtggtcgcttcaccaacttctcaaactgttaaatga

SEQ ID NO: 2 - DNA, 2163 bp, Arabidopsis thaliana
  complementary DNA encoding ABI3 protein
atgaaaagcttgcatgtggcggccaacgccggagatctggctgaggattgtggaatactcggtggagacgctgatgatactgttttgatggatggaattgatgaagttggtagagagatctggttagatgaccatggaggagataataatcatgttcatggtcatcaagatgatgatttgattgttcatcatgacccttcaatcttctatggagatctcccaacgcttcctgatttcccatgcatgtcgtcttcatcatcgtcttcaacatctccagctcctgtcaacgcaatcgtctcctcagcctcttcttcttcggcagcttcttcctccacttcctcagctgcttcttgggctatattgagatcagatggagaagatccgactccaaaccaaaaccaatacgcatcaggaaactgtgacgactcttctggtgcattgcaatccacagcttccatggagattccattagacagcagtcaaggttttggttgcggcgaaggcggtggtgattgcattgatatgatggagactttcgggtacatggatctacttgatagcaacgagttctttgacacctcagctatatttagccaagacgacgacacgcaaaaccctaacttgatggaccaaacccttgagagacaagaagaccaggtcgttgttccgatgatggagaataacagtggtggagacatgcaaatgatgaattcttccttggaacaggacgatgatctcgctgctgtgtttttggagtggctaaagaacaacaaggagactgtgtcggctgaggatttgaggaaagtaaagataaagaaagctacgattgaatcagcggcaagaagactaggcggtggtaaagaagcgatgaagcagcttttaaagctgattcttgaatgggtccaaactaatcacttacaaagaagacgcaccaccaccaccaccaccaacctctcttatcaacaatcattccaacaagatccatttcaaaaccctaaccctaataacaacaacctaatcccaccgtccgaccaaacctgtttctcaccttcaacatgggttcctccaccaccacaacaacaagcttttgtctcggacccgggttttggatacatgcctgctccaaactatccgccacagccagagttccttcctttacttgaatctccaccgtcatggccaccaccaccacagtctggtcccatgccacatcaacaattccccatgccgccaacctcgcagtataatcaatttggagatccaacaggtttcaatggatacaacatgaatccgtaccaatatccttatgttcctgcaggacaaatgagagatcagagattactccgtttgtgttcctcagcaactaaagaggcaagaaagaaacggatggcgagacagaggaggttcttgtctcatcaccacagacataacaacaacaacaacaacaacaacaataatcagcagaaccaaacccaaatcggagaaacctgtgccgcggtggctccacaacttaaccccgtggccacaaccgccacgggagggacctggatgtattggcctaatgtcccggcagtgccgcctcaattaccgccagtgatggagactcagttacctaccatggaccgagctggctcagcttctgctatgccacgtcagcaggtggtaccagatcgccggcagggatggaaaccagaaaagaatttgcggtttctcttgcagaaagtcttgaagcaaagcgacgtgggtaacctcggaaggatcgttttgccaaaaaaagaagctgagacacacttgccggagctagaggcaagagacggcatctctctggccatggaagacatcggaacctctcgtgtttggaacatgcgctacaggttttggcctaacaacaaaagcaggatgtatctcctcgagaacaccggcgattttgtgaaaaccaatgggctccaagaaggtgatttcatagtcatatactccgacgtcaaatgtggcaaatatttgatacgaggggttaaagtaagacaaccgagcggacaaaagccggaggccccaccgtcgtcagcagctacgaagagacaaaacaagtcgcaaaggaacataaacaataactctccgtcggcgaatgtggtggtcgcttcaccaacttctcaaactgttaaatga

SEQ ID NO: 3 - protein, 720 aa, Arabidopsis thaliana
  ABI3 protein
MKSLHVAANAGDLAEDCGILGGDADDTVLMDGIDEVGREIWLDDHGGDNNHVHGHQDDDLIVHHDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMMENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKCGKYLIRGVKVRQPSGQKPEAPPSSAATKRQNKSQRNINNNSPSANVVVASPTSQTVK